(19) United States
(12) Patent Application Publication
(10) Pub. No.: US 2024/0286985 A1
(43) Pub. Date: Aug. 29, 2024

(54) HETEROGENEOUS CATALAYST COMPRISING A TRANSITION METAL ON A SULFONIC ACID FUNCTIONALIZED SiO2 CARRIER AND THE USE OF SUCH CATALYST IN A PROCESSES FOR PRODUCING AN ETHER

(71) Applicants: Dow Global Technologies LLC, Midland, MI (US); The Board of Trustees of the University of Illinois, Urbana, IL (US)

(72) Inventors: Jing Luo, Midland, MI (US); Xue Chen, Manvel, TX (US); David G. Barton, Midland, MI (US); David W. Flaherty, Urbana, IL (US); Claudia Eugenia Berdugo-diaz, Urbana, IL (US); Yangsik Yun, Champaign, IL (US)

(21) Appl. No.: 18/681,921

(22) PCT Filed: Nov. 2, 2022
(86) PCT No.: PCT/US22/48647

§ 371 Date: Feb. 7, 2024

Related U.S. Application Data
(60) Provisional application No. 63/276,308, filed on Nov. 5, 2021.

(51) Int. Cl. C07C 41/18; B01J 23/44; B01J 31/02; B01J 31/28; B01J 35/30

(52) U.S. Cl. CPC C07C 41/18; B01J 23/44; B01J 31/0225; B01J 31/28; B01J 35/394; B01J 2231/643; B01J 2531/005; B01J 2531/824

(57) ABSTRACT

A process for producing an ether including treating (a) an ester with (b) hydrogen in the presence of (c) a heterogeneous catalyst to reduce the ester by hydrogenation to form an ether product, wherein the heterogeneous catalyst comprises a transition metal deposited on a sulfonic acid functionalized SiO₂ support.

### FIELD

The present invention relates to processes for producing an ether compound; and more specifically, the present invention relates to processes for producing an ether compound directly from an alkyl ester using molecular hydrogen on a heterogeneous catalyst.

### BACKGROUND

Ethers are used in various applications, including as a solvent. Ethers are particularly desirable for use as a solvent in applications because ethers possess excellent solvency, chemical stability and compatibility with other organic solvents and formulated products. Known routes of synthesizing ethers include the following three routes: (1) alkyl halides treated with alkoxides (so called the “Williamson ether synthesis”); (2) alcohol addition to an olefin; and (3) acid catalyzed coupling of alcohols. However, the above three routes have undesirable limitations including: (1) use of strongly acidic or basic conditions which can lead to competing elimination reactions that produce undesired olefins; (2) limited options of bio-sourced raw materials due to lack of reactivity with the above reactions which limit the structural variety of products; and (3) use of toxic raw materials and generation of waste streams in a manufacturing process. Therefore, what is desired is to provide a viable route for producing an ether that can be successfully scaled up commercially without the limitations of the above known routes.

For example, heretofore the known methods for producing an ether include the following: (1) a process using metal hydride/Lewis acid complexes, or hydrosilanes as stoichiometric hydride donors with precious metal catalysts as disclosed in J. Org. Chem., 2007, 72, 5920-5922; Tetrahedron Letters, 2017, 58, 3024-3027; (2) a process for producing a thionate (a salt or ester of thionic acid) such as a thioether (a sulfide which is a bonded compound of sulfur and two organic residues) as disclosed in J. Org. Chem., 1981, 46, 831-832; (3) a process for the catalytic reduction of α-monoglycerides with a 5 percent (%) Pd/C catalyst mixed with an acid cocatalyst at about 700 psi (4.8 megapascals [MPa]) and 120 degrees Celsius (° C.) as disclosed in U.S. Pat. No. 8,912,365; (4) a non-direct process for the hydrogenation of ethyl acetate into ethanol intermediates that subsequently couple to form a symmetrical ether by-product on Re/(gamma-Al₂O₃) or Re/(theta-Al₂O₃) at up to 4.6% conversion and 57% selectivity as disclosed in Russian Chemical Bulletin 1988, 37(1), 15-19 and Russian Chemical Bulletin 1986, 35, 280-283; and (5) a process for the hydrogenation of lactone to cyclic ethers, for example (e.g.), for production of tetrahydrofuran, in high selectivities such as greater than (>) 90% using various metal catalysts on various support carriers as disclosed in U.S. Pat. Nos. 3,370,067; 3,894,054; and 4,973,717 (6) a process using homogeneous metal complex catalysts (e.g. a ruthenium/triphos complex) which require impractical separation of catalyst from product. Angewandte Chemie, International Edition, 2015, 54, 5196-5200; ChemSusChem, 2016, 9, 1442-1448.

In a recently filed patent application, U.S. 63/107,739, a process to directly convert ester to ether with transition metals supported by metal oxide supports, such Nb₂O₅ and WO₃ is described. The invention reported high direct selectivity of ether formation via hydrogenation, however, the ether product absolute selectivities from hydrogenation were generally low, typically in a range of from 5 to 10% with a maximum value of 16%. Further improvement of the catalyst selectivity is required in order to make the process economically valuable.

It would therefore be desirable to have alternative processes for producing ethers that can be commercially manufactured and that provide advantages over existing processes, including improved absolute and/or direct selectivity.

### SUMMARY

The present invention is directed to new processes for producing an ether product from an ester starting raw material using transition metal catalysts on sulfonic acid functionalized SiO₂ carriers.

In a broad embodiment, a process of the present invention includes producing an ether by hydrogenation of an ester in the presence of a heterogeneous catalyst.

In one embodiment, a process of the present invention includes direct selective reduction of carboxylic acid derivatives into ethers using molecular hydrogen and a proper catalyst formulation for achieving a high (e.g., >10%) absolute ether selectivity with a high (e.g., >80%) direct ether selectivity. Absolute ether selectivity is the percentage of the total products formed in the reaction, while the direct ether product selectivity is the percentage of direct ether product over the total ether products.

In another embodiment, a process of the present invention for producing an ether comprises mixing: (a) at least one ester with (b) hydrogen in the presence of (c) a heterogeneous catalyst to reduce the ester by hydrogenation to form an ether.

In still another embodiment, the present invention includes a solvent comprising the above ether product produced by the above process.

Some of the advantageous features that can be provided by one or more embodiments of the process of the present invention include, for example:
(1) An active catalyst is used in a one-step process. The catalyst is active for direct hydrogenation of an ester to reduce the ester to form an ether, rather than going through a known two-step ether formation process such as (i) ester hydrogenolysis to form alcohol followed by (ii) alcohol dehydration.
(2) A relatively inexpensive route is used. The process employs inexpensive molecular hydrogen as a reduction agent, rather than employing expensive hydrosilanes, metal hydrides, or metal hydride/Lewis acid complexes as a hydride donor. The highly reactive hydrosilanes or metal hydride employed in prior art processes also requires the design of a complicated and expensive process to ensure the safety of the operators running the prior art processes.
(3) A heterogeneous catalyst is used. The catalyst being heterogeneous rather than homogeneous, can contribute to lower manufacturing costs due to catalyst recyclability and separation.
(4) An efficient process is used.
(5) A flexible process is used. The process is applicable to a general ester compound (either cyclic or acyclic) as a feed material; and the process is not limited to a specified ester compound.

### DETAILED DESCRIPTION

In one embodiment, the present invention includes a distinct and novel method for synthesizing ethers from esters with heterogeneous catalysts. The reactions of ester on heterogeneous catalysts include various chemical reaction routes or pathways, for example, hydrogenolysis, hydrolysis, dehydration hydrogenation, and transesterification. In a general embodiment, the process of the present invention includes producing an ether by hydrogenation of an ester, such as a propyl acetate, in the presence of a heterogeneous catalyst. The present invention's novel hydrogenation reaction pathway or scheme, for example, the hydrogenation of propyl acetate reduction reaction scheme with R₁ being —CH₃ and R₂ being —CH₂CH₃, is generally illustrated as Reaction Scheme (I) as follows:

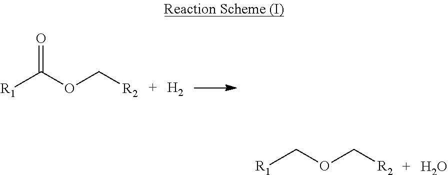

In the above Reaction Scheme (I), water is generated by the reduction process; and the generated water can be separated by conventional processes such as distillation or other procedures known in the art. Functional groups R₁ and R₂ can be alkyl functional groups including straight or branched-chain alkyl groups, cyclic or non-cyclic alkyl groups; and mixtures thereof. Examples of the esters herein include but are not limited to ethyl acetate, propyl acetate, butyl acetate, ethyl propionate, butyl propionate and mixtures thereof. The desired ether product resulting from the above Reaction Scheme (I) can be a symmetric ether when R₁ is equivalent to R₂; or an unsymmetric ether when R₁ is not equivalent to R₂, for example, the unsymmetric ether can be ethyl propyl ether.

A “symmetric ether” herein means an ether that contains two identical functional groups, wherein R₁ is identical to R₂. An “unsymmetric ether” herein means an ether that contains two different functional groups, where R₁ is not identical to R₂.

In the present invention the desired reaction scheme, Reaction Scheme (I), is a direct hydrogenation route to obtain the desired ether product. By “direct hydrogenation” or “direct selective reduction” it is meant that carbonyl oxygen is removed from ester (R₁COOCH₂R₂) by hydrogenation to form ether (R₁CH₂OCH₂R₂) while maintaining the alkoxyl group intact. The present invention process is different from known processes because the present invention process does not undergo a typical route for ester (R₁COOCH₂R₂) hydrogenation, where ester first breaks into two alcohols (R₁CH₂OH+R₂CH₂OH molecules via hydrogenolysis and then subsequently forms a mixture of ethers (R₁CH₂OCH₂R₁+R₁CH₂OCH₂R₂+R₂CH₂OCH₂R₂) via dehydration. Direct hydrogenation can maintain the structure of the ether from ester by only eliminating the carbonyl oxygen. Thus, an unsymmetric ester provided to this process advantageously results in the direct production of an unsymmetric ether because this process does not break the ester into two alcohol molecules via hydrogenolysis. The selectivity of ethyl propyl ether in the reaction examples are listed in Table III and Table V described below in the Examples.

The term “direct ether product” herein means an ether that is formed by a one-step reduction process of ether from ester.

The term “indirect ether product” herein means an ether that is formed by a two-step reduction process of ether from ester including the steps of: (i) hydrogenolysis and (ii) dehydration.

The term “direct ether product selectivity” herein means the percentage of the direct ether product over the total ether products. For example, for the reduction of propyl acetate, ethyl propyl ether is the direct ether product, and the direct ether product selectivity is the percentage of ethyl propyl ether over the total ether products (ethyl propyl ether+dipropyl ether+diethyl ether).

The term “ether product absolute selectivity” herein means the percentage of the ether product in the total products formed in the reaction (e.g., ethers, alcohols, and alkanes).

Advantageously, one unique factor of the present invention includes an increase in direct ether selectivity using the one-step process of the present invention versus the known two-step process.

In one embodiment, a process of the present invention for producing an ether comprises treating (a) an ester with (b) hydrogen in the presence of (c) a heterogeneous catalyst to reduce the ester by hydrogenation to form an ether.

In one desirable embodiment and as shown in Reaction Scheme (I) above, a process of the present invention for producing an ether comprises the steps of: (A) feeding into a reactor, an ester compound, component (a), such as propyl acetate; (B) feeding into the reactor, hydrogen, component (b), to form a hydrogen atmosphere in the reactor; and (C) charging the reactor with a heterogeneous catalyst system, component (c), comprising a transition metal on a sulfonic acid functionalized SiO₂ support; sufficient to generate a hydrogenation reaction in the reactor; and (D) heating the contents of the reactor, components (a)-(c), at a temperature sufficient to reduce the ester compound to form an ether compound. For example, the heating step (D), can take place at a temperature of from 350 Kelvin (K) to 650 K. It will also be readily understood by those in the art that the order of the recited steps can be altered in certain circumstances, or steps may be done simultaneously

As is generally known in the art a “heterogeneous catalyst” refers to a catalyst whose phase (e.g., solid, liquid or gas) is different from the phase of the reactants. For example, the reactants (esters and hydrogen) and product (e.g., ether) can be in the liquid or gas phase while the catalyst is a solid.

In the present invention, the heterogeneous catalytic system of the present invention is a combination of a transition metal and a sulfonic acid functionalized SiO₂ support. For instance, the catalyst used in some embodiments of the present invention can include from 0.1 weight percent (wt %) to 20 wt % of a transition metal, preferably 0.5 to 15, or even 1 to 10 percent by weight, supported on sulfonic acid functionalized SiO₂.

As is generally known in the art, sulfonic acid functionalized SiO₂ carriers can be produced by contacting conventional silica (SiO₂) carriers with a reagent comprising sulfonic acid groups, such as tosic acid (or p-toluensulfonic acid) or propylsulfonic acid under conditions to allow at least some of the groups to be converted to sulfonic acid-containing groups. The aromatic ring of the tosic acid makes it slightly more acidic than alkylsulfonic acids such as propylsulfonic acid, however, tests have demonstrated that they aromatic and alkyl based sulphonic acids have comparable strength. Suitable sulphonic functionalized SiO₂ material for use as carriers in the present invention can be obtained from commercial sources such as SiliCycle under the trade names SiliaBond™ Propylsulfonic Acid (SCX-2) and SiliaBond™ Tosic Acid (SCX), or can be prepared by methods generally known in the art.

For the carriers of the present invention, it is believed that acidity originates from a proton attached to the sulfonic acid group of p-Toluenesulfonic acid; hence, the sulfur content is used as a proxy for counting acid sites. The molar ratio of transition metal to sulfur for the present invention can be in a range from 0.1 to 1, preferably from 0.15 to 0.75, or even 0.2 to 0.5

The synergistic effect from both the transition metal component and the sulfonic acid functionalized SiO₂ support component promotes the direct ether reduction route, as described in the reaction pathway Reaction Scheme (I). Without the combination of a transition metal and a sulfonic acid functionalized SiO₂ support member as disclosed herein, an undesirable two-step ether formation route would take place. Steady-state rates and product selectivity for competing reaction pathways for a model ester compound are obtained in, for example, a packed-bed reactor and/or a trickle bed reactor as functions of reactant pressures, temperature, and ester conversion, which are controlled via the surface residence time.

The ester compound, component (a), to be reduced to an ether, can include one or more ester compounds, including, for example, a carboxylic acid derivative; an ester containing straight or branched-chain alkyl groups, and cyclic or non-cyclic alkyl groups; and mixtures thereof. In some embodiments, an unsymmetric ether includes a Reaction Scheme (I) wherein the R₁ groups are not equal to the R₂ groups. In some embodiments, the ester useful in the present invention can be, for example, propyl acetate (available from Sigma-Aldrich); butyl acetate (available from Sigma-Aldrich); butyl propionate, glycerin ester; and mixtures thereof.

The concentration of the ester, component (a), is not particularly critical. However, it may be advantageous, in some embodiments, for the ester to be present in an amount of at least 1 wt % so as to provide a desirable production rate and/or avoid an increase in separation costs. In some embodiments, the concentration of the ester is from 1 wt % to 100 wt %. The concentration of the ester is based on the total weight of ester compounds in the liquid feed raw material.

The concentration of the hydrogen, component (b), useful in the process of the present invention includes, for example, from 3 wt % to 100 wt % in one embodiment, from 10 wt % to 100 wt % in another embodiment, and from 50 wt % to 100 wt % in still another embodiment. Hydrogen with a low concentration of, for example less than (<) 3 wt % may decrease the reactivities or ether selectivities; and therefore, in such a case an undesirable increase of the reaction pressure would be required. The concentration of the hydrogen is based on the total weight of hydrogen in the gas feed raw material.

In a broad embodiment, the catalyst used in the process, component (c), of the present invention, can include one or more heterogeneous catalyst compounds. The catalysts used in the present invention process include, for example, a combination of (ci) a transition metal, supported on (cii) a sulfonic acid functionalized SiO₂ support (carrier) member. For example, the transition metal (component (ci)) may include palladium (Pd); platinum (Pt); ruthenium (Ru); cobalt (Co); copper (Cu); rhodium (Rh); rhenium (Re); nickel (Ni); and mixtures thereof, with palladium being preferred in some applications. The acidic sulfonic acid functionalized SiO₂ carrier member (component (cii)) can be any sulfonic acid functionalized SiO₂ carrier or mixture of sulfonic acid functionalized SiO₂ carriers.

The heterogeneous catalyst of the present invention exhibits some advantageous properties. For example, the heterogeneous catalysts useful in the present invention provide a synergistic effect between the metallic compound (e.g., Pd or Pt) of the catalyst and the sulfonic acid functionalized SiO₂ carriers of the catalyst in order to catalyze direct ester hydrogenation. Otherwise, ether selectivities may decrease.

The heterogeneous catalyst, component (c), includes, for example, from 0.01 wt % to 20 wt % of the metallic compound based on the total weight of the heterogeneous catalyst in one embodiment, from 0.1 wt % to 10 wt % of the metallic compound based on the total weight of the heterogeneous catalyst in another embodiment, and from 1 wt % to 5 wt % of the metallic compound based on the total weight of the heterogeneous catalyst in still another embodiment. The process equipment used to carry out the reduction process can be any conventional reactor such as a packed-bed reactor or a trickle bed reactor. And, the ester conversion and ether selectivities can be controlled via the reactor pressure, temperature, and surface residence time.

For example, the pressure of the process of the present invention is from 0.1 MPa to 10 MPa in one embodiment, from 2 MPa to 6 MPa in another embodiment, and from 6 MPa to 10 MPa in still another embodiment. Below the aforementioned pressure range may lead to lower reactivities or lower ether selectivities than disclosed herein. A pressure higher than the aforementioned pressure range may be sufficient to use in the present invention; however, it may require a higher cost in reactor construction and operation.

For example, the temperature of the process of the present invention is from 350 K to 650 K in one embodiment, from 400 K to 500 K in another embodiment, and from 500 K to 650 K in still another embodiment. Below the aforementioned temperature range may lead to lower reactivities than disclosed herein. A temperature higher than the aforementioned temperature range may lead to unwanted alkane and alcohol by-products; and therefore, in such a case the selectivities of the ether may decrease.

For example, the ester conversion of the process of the present invention is from 1% to 100% in one embodiment, from 1% to 50% in another embodiment, and from 50% to 100% in still another embodiment. In some embodiments, ester conversions higher than the aforementioned conversion range may lead to more side reaction products.

The process of the present invention may be carried out as a batch process or a continuous process. When using a batch process, in some embodiments, the residence time of the process of the present invention is, for example, from 0.1 hour (hr) to 24 hr in one embodiment, from 0.1 hr to 8 hr in another embodiment, and from 1 hr to 24 hr in still another embodiment. In some embodiments, residence times below the aforementioned residence time range may lead to a lower ester conversion; and in some embodiments, residence times above the aforementioned residence time range may lead to unwanted side reaction products.

When using a continuous process, in some embodiments, the residence time of the process of the present invention is, for example, from 0.1 second (s) to 100 s in one embodiment, from 1 s to 10 s in another embodiment, and from 10 s to 100 s in still another embodiment. Residence times below the aforementioned residence time range may lead to a lower ester conversion; and in some embodiments, residence times above the aforementioned residence time range may lead to unwanted side reaction products.

Some advantageous properties and/or benefits of using the reduction process of the present invention include, for example, the process of the present invention can achieve steady-state rates; and the process can provide better selectivities of product for competing reaction pathways for an ester compound, even better than other heterogeneous catalysts that do no feature sulfonic acid functionalized SiO₂ supports. Also, conventional processes for producing an ether also produces salt whereas the process of the present invention does not generate salt.

After an ester compound undergoes the reduction process, the resulting ether product is formed. The turnover rate of ester to the ether product can be from 10⁻⁸ moles of ether per gram catalyst per second (mol/g_{cat}·s) to 105 mol/g_{cat}·s in one general embodiment, from 5×10⁻⁸ mol/g_{cat}·s to 5×10⁻⁶ mol/g_{cat}·s. Ester turnover rates below the aforementioned rate range may lead to a lower ether production rate; and in some embodiments, ester turnover rate above the aforementioned residence time range may lead to unwanted side reaction products.

The selectivity of the ether product can depend on whether a vapor process or liquid process is used to form the ether and whether a batch process or continuous process is used. In general, the selectivity of the direct ether product is >10% in one embodiment, from 10% to 25% in another embodiment, and from 25% to 50% in still another embodiment.

While the ether product produced by the process of the present invention can be a symmetric ether or an unsymmetric ether, as an illustration of the present invention and not to be limited thereby, the present invention process is described with reference to an unsymmetric ether. It has been surprisingly discovered that the process of the present invention is selective for unsymmetric ether because in the present invention process the ester is directly converted to ether, without undergoing ester hydrogenolysis and alcohol dehydration. Ester hydrogenolysis and alcohol dehydration are two processes that are known to not be selective for a specific ether. If an unsymmetric ether is desired for a specific process or end use, then use of the present invention process is more advantageous than conventional processes because:
(1) An ether product is more stable than the corresponding ester product under basic and acid conditions. Also, the ether products of the present invention do not typically undergo hydrolysis which can occur at high humidity and/or high temperatures.
(2) It is believed that the hydrogen reaction chemistry of the present invention process maintains the backbone of the ester product intact. During the hydrogenation, only the oxygen molecule is broken away from the backbone which leaves the carbon molecules and backbone oxygen intact. In conventional reaction processes, the reaction breaks the backbone and combines parts back together under different reaction conditions. Thus, no direct hydrogenation/reduction of the ester to an ether occurs.
(3) The process of the present invention minimizes undesirable side reactions that may detrimentally affect the selectivities of the desired ether product.

The ether product of the present invention has a minimal impact on the environment, since the ether product is derived from organic and renewable sources. For example, the ether product advantageously can be used as a global green and bio-based solvent to address the stringent regulations imposed on chemical-based industrial solvents in relation to toxicity, non-biodegradability, volatile organic compound (VOC) emissions, and the like. Green and bio-based solvents are typically used in paints and coatings applications. Other applications include adhesives, pharmaceuticals, and printing inks. In some embodiments, the ether product can be used as a foam control agent and a flavor additive. In other embodiments, the ether product can be used in cosmetics and personal care applications.

The present invention provides biobased solvents at a cost and performance advantage to known solvents in the industry. In addition, the chemical transformation provided by the present invention process could be useful to produce, for example, bio-based surfactants, defoamers and lubricants with both an economically and environmentally favorable process.

The ether generation process of the present invention can also be used to develop: (1) a more robust capping process to overcome the issues of limited reactant alkyl chloride types and final product impurities; (2) new capped low viscosity-low volatility lubricants; and (3) new surfactants and new biobased defoamers for food and pharmacy applications, metalworking fluids applications, and other applications utilizing an ether solvent.

### EXAMPLES

The following Inventive Examples (Inv. Ex.) and Comparative Examples (Comp. Ex.) (collectively, “the Examples”) are presented herein to further illustrate the present invention in detail but are not to be construed as limiting the scope of the claims. Unless otherwise stated all parts and percentages are by weight.

### Catalysts

Commercial silica (SiO₂) functionalized with sulfonic acid groups is used as support of palladium species. This support (SiO₂-Tosic Acid, SiO₂-TA, SiliCycle, 0.78-0.81 meq g⁻¹) is used to scavenge metals from organic solutions and few publications use similar synthesis techniques for preparation of Pd deposited on functionalized SiO₂. (Green Chemistry 2010, 12 (7), 1163-1166; ChemCatChem 2015, 7 (14), 2085-2094). For these examples, catalysts are prepared by the methanol-mediated weakly-capping growth approach (WCGA) method (Journal of the American Chemical Society 2015, 137 (36), 11743-11748; Industrial & Engineering Chemistry Research 2021, 60, 2326-2336). First, palladium acetate (Pd(OAc)₂, Sigma Aldrich, 99.98%) is dissolved in methanol (Fisher Chemical, HPLC Grade) to achieve a concentration of 170 mg Pd L⁻¹ while stirring with an overhead stirrer. After the precursor was dissolved, SiO₂-TA is added to the solution and left stirring for more than 18 hours. The initial yellow solution turns dark during the synthesis. Pd—SiO₂-TA solids are recovered by filtration and washed with methanol. After filtration, the solid has a dark red-black color and the filtrate is black colored. Such black colored filtrate indicates incomplete uptake of Pd. The samples are dried in a static oven at 353 K for more than 18 h and then treated with flowing 20 kPa H₂ in He at 403 K (5 K min⁻¹) for 2 hours. Elemental analysis by Inductively Coupled Plasma (ICP) gives the ratio of Pd to H⁺ (S).

The catalysts for Comparative Examples 4 to 7 in Table 1 are prepared using the incipient wetness impregnation method as described in a recently filed patent application, U.S. 63/107,739 using the materials described in Table 1.

The catalyst formulations used in the examples are listed and further described in Table I.

TABLE I
Catalyst Formulations

| | | | | Pd | S | Molar |
|---|---|---|---|---|---|---|
| Catalyst | | | | Weight | Weight | Ratio |
| No. | Metal | Precursor | Support | (%) | (%) | of Pd/S |
| 1 | Pd | Pd(OAc)₂ | SiliaBond | 1.03 | 1.83 | 0.17 |
| (inventive) | | | Tosic Acid | | | |
| 2 | Pd | Pd(OAc)₂ | SiliaBond | 2.27 | 1.75 | 0.39 |
| (inventive) | | | Tosic Acid | | | |
| 3 | Pd | Pd(OAc)₂ | SiliaBond | 5.12 | 2.15 | 0.72 |
| (inventive) | | | Tosic Acid | | | |
| 4 (comp.) | Pd | Pd(NO₃)₂ | Nb₂O₅ | 0.8 | N/A | N/A |
| | | 2H₂O | | | | |
| 5 (comp.) | Pd | Pd(NO₃)₂ | WO₃ | 1 | N/A | N/A |
| | | 2H₂O | | | | |
| 6 (comp.) | Pd | Pd(NO₃)₂ | Al₂O₃ | 1 | N/A | N/A |
| | | 2H₂O | | | | |
| 7 (comp.) | Pd | Pd(NO₃)₂ | SiO₂ | 1 | N/A | N/A |
| | | 2H₂O | | | | |

Examples using Cat. 1-Cat. 3 are representative Inventive Examples (Inv. Ex.) of the present invention, while the Examples using Cat. 4-Cat. 7 are Comparative Examples (Comp. Ex.).

### Test Measurements

The process of the present invention comprises a process that: (1) uses molecular hydrogen (H₂) as a reducing agent; (2) is conducted in vapor phase or liquid; and (3) uses a heterogeneous catalyst for ester reduction to ether, where the heterogeneous catalyst includes a transition metal, for example a Pd-based catalyst, and where the catalyst support (carrier) is a sulfonic acid functionalized SiO₂ for the inventive examples or an acid support, for example, a WO₃-based catalyst carrier or a Nb₂O₅-based catalyst carrier, for the comparative examples.

### Part A: Catalytic Rate Measurements in Vapor-Phase Reactor

Cat. 1-3, and comparative Catalysts 4 and 5 are tested in a vapor-phase reactor under the conditions reported in Table II. The vapor-phase reactor is a tubular packed bed reactor held within a stainless-steel tube (9.5 millimeters [mm] outer diameter [O.D.]) containing from 10 milligrams (mg) to 200 mg of catalyst. The catalyst is held at the center of the reactor using glass rods and packed glass wool. The tubular reactor is placed within a three-zone furnace (available from Applied Test Systems, 3210) that was controlled by an electronic temperature controller (available from Watlow, EZ-Zone). The catalyst temperature is measured by a K-type thermocouple contained within a 1.6 mm stainless-steel sheath (available from Omega) that is coaxially aligned within the reactor and submerged within the catalyst bed. The volume of the catalyst bed is kept constant at 1.4 cubic centimeters (cm³) of material by mixing excess silicon carbide (SiC) (available from Washington Mills, Carborex green 36) with the desired amount of catalyst. The system is pressurized using a back-pressure regulator (BPR, an Equilibar LF Series available from Equilibar Precision Pressure Control) which is controlled by an electronic pressure regulator (EPR, an Equilibar GP1 available from Equilibar Precision Pressure). The reactor pressure is monitored upstream and downstream of the catalyst bed using a digital pressure gauge (available from Omega) and the EPR, respectively.

The gases used in the Examples are: H₂ (available from Airgas Inc. as “Ultra High Purity 5.0”) and He (available from Airgas Inc. as “Ultra High Purity 5.0”). The gas flow rates are controlled using mass flow controllers (available from Bronkhorst as “EL-FLOW High Pressure”). The flow rate of liquid propyl acetate (C₅H₁₀O₂, supplied by Sigma Aldrich, 537438, at greater than or equal to [≥] 99.5%) is controlled using a stainless-steel syringe pump with a Hastelloy cylinder (100DX with D-series controller, available from Teledyne Isco) as the C₅H₁₀O₂ is fed through a polyether ether ketone (PEEK) polymer tube (1.6 mm O.D. and 0.25 mm inner diameter [I.D.]) the exit of which is positioned within a small bed of non-porous sand (SiO₂ 50-70 mesh particle size, supplied by Sigma Aldrich, 274739) within a cross flow of H₂. The transfer lines surrounding the liquid inlet are kept at 373 K using heating tape (available from Omega) to avoid condensation. All transfer lines downstream of the liquid inlet are heated above 373 K using heating tape; and the line temperatures are monitored with K-type thermocouples (available from Omega) displayed on a digital reader (available from Omega).

Catalyst is pretreated in situ by heating the catalyst to the desired temperature at 0.08 Kelvin per second (K s⁻¹) and holding the catalyst at that temperature for the desired time within 101 kilopascals (kPa) flowing H₂ at 100 cubic centimeters per minute (cm³ min⁻¹) prior to all catalytic measurements. The effluent of the reactor is characterized using on-line gas chromatography (HP 6890, available from Agilent). The gas chromatograph (GC) is equipped with a capillary column (DB-624 UI, 30 meters (m) length, 0.25 mm I.D., 1.40 micron [μm]) connected to a flame ionization detector to quantify the concentrations of combustible species. Sensitivity factors and retention times for all components are determined using gaseous and liquid standards. Control of the reactor pressure and temperature, reactant flowrates, and the GC sampling are automated to allow for continuous measurements. Conversions are calculated on a carbon basis based on the amount of carbon that appears in the products. The carbon and oxygen balance closes within ±20%. Reactor conditions during rate and selectivity measurements are varied by sequentially decreasing and then increasing the reactant pressure over the full range of 1 MPa to 10 MPa such that one or more of the conditions is measured at least twice throughout the experiment to ensure that measured trends were not a result of systematic deactivation.

### Part B: Catalytic Rate Measurements in Liquid-Phase Reactor

Cat. 3 and Comparative catalysts 6 and 7 are tested in a liquid-phase reactor. Rate and selectivity measurements are performed in a trickle bed reactor comprising a stainless-steel tube (1.6 mm OD) containing 1,000 mg to 4,000 mg of catalyst (30 mesh to 60 mesh), which is held at the center of the reactor using Pyrex glass rods and packed glass wool. The reactor is heated with an aluminum clamshell including two heat cartridges that was controlled by an electronic temperature controller (an EZ-Zone available from Watlow). The reaction temperature is measured by a K-type thermocouple contained within a 3.2 mm stainless-steel sheath (available from Omega) that is coaxially aligned within the reactor and submerged within the aluminum clamshell. The system is pressurized up to 6.6 MPa using a dome loaded back pressure regulator (BPR), an Equilibar LF Series, available from Equilibar Precision Pressure Control which is controlled by an electronic pressure regulator (EPR), an Equilibar GP1 available from Equilibar Precision Pressure Control. The reactor pressure is monitored using a digital pressure gauge (available from Omega) and the EPR.

The gas flow rates of H₂ (available from Airgas); and He (available from Airgas); are controlled using mass flow controllers (EL-FLOW High Pressure controllers available from Bronkhorst). The flow rate of liquid propyl acetate (C₅H₁₀O₂) supplied by Sigma Aldrich, 537438, at ≥99.5%, is controlled using a high performance liquid chromatography (HPLC) pump (P-LST40B available from Chromtech) as the C₅H₁₀O₂ fed through a stainless-steel tube (1.6 mm O.D. and 0.15 mm I.D.) within a cross flow of H₂ and He.

Catalysts are pretreated in situ by heating to 403 or 503 K at 0.08 K s⁻¹ and holding for 1 hr within flowing He (20 kPa) and H₂ (81 kPa) at 50 cm³ min⁻¹ prior to all catalytic measurements. The effluent of the reactor passes through a stainless-steel cooling chamber including cold water (at a temperature of ˜377 K), and then gas and liquid products are separated in a gas-liquid separator (GLS). The liquid products collected in the GLS are delivered by an HPLC pump to a high-pressure liquid sampling valve (LSV, Transcendent Enterprise Inc., PLIS-6890, 1 μL of injection volume) which is attached to on-line gas chromatography (Agilent, HP 7890B). At outlet of LSV, manual BPR (Swagelok) is installed so that the pressure of the liquid is maintained at 1,380 kPa to prevent the products from evaporation in the sampling system. The gas and liquid products are characterized using on-line gas chromatography (Agilent, HP 7890B). The GC is equipped with two capillary columns (DB-Wax UI, 60 m length, 0.25 mm I.D., 0.25 μm, available from Agilent) for liquid product, and a GS-GASPRO (a GC column, 60 m length, 0.32 mm I.D., available from Agilent) is connected to a flame ionization detector to quantify the concentrations of species. Sensitivity factors and retention times for all gas products and liquid products are determined using gaseous standards and a methanizer (Polyarc System, PA-SYC-411, available from Activated Research Company), respectively. Control of the reaction pressure and temperature, reactant and product flowrates, and the GC samplings are automated to allow for continuous measurements. Conversions are calculated on a carbon basis based on the amount of carbon that appears in the products. The carbon balance closes within ±10%.

### Test Results

### Reaction Pathway Using Propyl Acetate as the Ester Starting Compound

A kinetic investigation of the catalysts (Cat. 1-Cat. 3) used in the Examples for the direct selective reduction of propyl acetate (an example of a representative ester) with molecular hydrogen is carried out to determine the reaction pathway for converting propyl acetate to an ether. There are several products observed in the product stream coming out of fixed bed reactor via GC analysis. The various products include, for example, light hydrocarbons such C₂-C₃ alkane and alkene, ethanol, propanol, dipropyl ether, diethyl ether, ethyl propyl ether, acetic acid, and ethyl acetate. Based on this observation, it can be concluded that the reaction took place via several routes in the reactor including for example: (1) hydrogenolysis of propyl acetate to one ethanol and one propanol; (2) hydrolysis of propyl acetate, to from propanol and acetic acid; (3) the alcohols can then undergo dehydration to form light hydrocarbons, as well as dehydration to form ether products, like dipropyl ether, diethyl ether and ethyl propyl ether; (4) transesterification of propyl acetate with ethanol to form ethyl acetate; and (5) the route of the present invention, Reaction Scheme (I), i.e., direct hydrogenation of propyl acetate with hydrogen to form ethyl propyl ether. Since the desired product is the ethyl propyl ether via direct hydrogenation of propyl acetate, the route of Reaction Scheme (I) of the present invention is the desired reaction pathway. It should be noted that ethyl propyl ether could also be formed from the above route (3). However, the route (3) above is not desired since route (3) above includes alcohol dehydration reactions, and such alcohol dehydration reactions are not selective for the formation of unsymmetric ether over symmetric ethers such as dipropyl ether or diethyl ether. While the present invention process is not limited to producing either a symmetric ether or an unsymmetric ether; advantageously, the present invention process, selectively and in a direct pathway, provides an unsymmetric ether when desired or needed.

### Part A: Vapor-Phase Ester Reduction Results

### Examples 1-3a and Comparative Examples 4-5

The results of the vapor phase and liquid phase are presented in Table II. In the vapor phase process, the selectivities for dipropyl ether and diethyl ether approach almost zero, indicating that substantially all the ethyl propyl ether is formed based on a direct hydrogenation reaction pathway, since the alcohol dehydration has no preference in selectivities for symmetric or unsymmetric ether. Therefore, the results of Inv. Ex 1˜3 suggest that Pd—SiO₂-TA is able to catalyze direct hydrogenation of ester to ether.

The results of Inv. Ex 1˜3 also indicate that all the three Pd—SiO₂-TA catalysts with various Pd loading, and Pd/S molar ratio can provide ethyl propyl ether absolute selectivities>25% and direct ether>90% in most cases, with a few exceptions at certain reaction conditions. Such high performance in ether selectivity has significantly surpassed the performance of the comparative examples on catalysts using metal oxide supports. The general absolute selectivities of ethyl propyl ether in previous patent application were reported in an average of 5˜10%.

### Part B: Liquid-Phase Ester Reduction Results

### Examples 3b and Comparative Examples 6-7

The results of Inventive Example 3b indicate that Pd—SiO₂-TA catalyst is somewhat less effective in providing a direct hydrogenation route of ester to ether in liquid-phase reactor. Although the same catalyst has been demonstrated to be effective in vapor-phase reactor (Inv. Ex 3a), the ethyl propyl ether absolute selectivities were significantly dropped below 10%, and the direct ether selectivities were decreased to 40˜80% due to the formation of dipropyl ether. This inventive example suggests that optimized reaction conditions are required for Pd—SiO₂-TA catalyst to achieve high-performance in ester reduction to ether.

The results of Comparative Examples 7 show that catalyst on SiO₂ supports are not effective in direct ester hydrogenation. It is indicating that the sulfonic acid functionalized SiO₂ support is important to catalyze direct ester hydrogenation.

TABLE II
Conversions and Product Selectivities

| | | | | | | | Absolute | Absolute | Direct |
|---|---|---|---|---|---|---|---|---|---|
| | | Feed Rate | | H₂ | C₅H₁₀O₂ | | Selectivity (% | Selectivity | Ether |
| Example | Catalyst | (mol C₅H₁₀O₂) · | Temperature | Pressure | Pressure | Conversion | Ethyl Propyl | (% Dipropyl | Selectivity |
| No. | No. | (gcat · s)⁻¹ | (K) | (kPa) | (kPa) | (%) | Ether) | Ether) | (%) |
| Inv. Ex. 1 | 1 | 2.4 × 10⁻⁶ | 401 | 6190 | 10 | 0.78 | 33.7 | 1.4 | 96.0 |
| (vapor | | | 388 | 6190 | 10 | 0.30 | 29.7 | 2.6 | 92.0 |
| phase) | | | 378 | 6190 | 10 | 0.13 | 25 | 3.7 | 87.1 |
| Inv. Ex. 2 | 2 | 3.4 × 10⁻⁶ | 401 | 6190 | 10 | 0.43 | 34 | 0.4 | 98.8 |
| (vapor | | | 388 | 6190 | 10 | 0.20 | 29.7 | 0.8 | 97.4 |
| phase) | | | | | | | | | |
| Inv. Ex. 3 a | 3 | 3.4 × 10⁻⁶ | 401 | 6190 | 10 | 1.56 | 25.7 | 0.7 | 97.3 |
| (vapor | | | 389 | 6190 | 10 | 0.73 | 20.9 | 1.1 | 95.0 |
| phase) | | | 376 | 6190 | 10 | 0.40 | 16.7 | 2.1 | 88.8 |
| Inv. Ex. 3b | 3 | 5.7 × 10⁻⁶ | 403 | 6204 | 207 | 0.70 | 8.5 | 2.1 | 80.2 |
| (Liquid | | | 383 | 6293 | 119 | 0.30 | 7.7 | 2.3 | 77.0 |
| phase) | | | 373 | 6324 | 88 | 0.10 | 5.2 | 7.2 | 41.9 |
| Comp. Ex 4 | 4 | 2.02 × 10⁻⁵ | 503 | 6190 | 10 | 1.04 | 4.4 | 0.0 | 98.2 |
| (vapor | | 1.01 × 10⁻⁵ | 503 | 6190 | 10 | 1.19 | 3.8 | 0.0 | 95.7 |
| phase) | | 3.75 × 10⁻⁶ | 503 | 6190 | 10 | 7.7 | 4.6 | 0.0 | 97.7 |
| | | 1.88 × 10⁻⁶ | 503 | 6190 | 10 | 12.0 | 4.3 | 0.0 | 97.7 |
| | | 6.74 × 10⁻⁷ | 503 | 6190 | 10 | 32.7 | 8.5 | 0.0 | 98.5 |
| | | 3.39 × 10⁻⁷ | 503 | 6190 | 10 | 47.8 | 7.5 | 0.0 | 97.3 |
| Comp. Ex. 5 | 5 | 8.36 × 10⁻⁶ | 503 | 6190 | 10 | 32.4 | 13.6 | 0.0 | 96.5 |
| (vapor | | | | | | | | | |
| phase) | | | | | | | | | |
| Comp. Ex 6 | 6 | 9.66 × 10⁻⁶ | 503 | 4977 | 1573 | 0.9 | 0.9 | 0.0 | 2.8 |
| (liquid | | | | | | | | | |
| phase) | | | | | | | | | |
| Comp Ex. 7 | 7 | 4.89 × 10⁻⁶ | 503 | 4977 | 1573 | 0.5 | 1.0 | 0.0 | 35.7 |
| (liquid | | | | | | | | | |
| phase) | | | | | | | | | |

What is claimed is:

1. A process for producing an ether product by direct selective reduction of an ester comprising treating (a) at least one ester with (b) hydrogen and in the presence of (c) a heterogeneous catalyst to directly and selectively reduce the at least one ester by hydrogenation to form the at least one ether; wherein the heterogeneous catalyst comprises a transition metal deposited on a sulfonic acid functionalized SiO₂ carrier.

2. The process of claim 1, wherein the ester is an ester containing straight or branched-chain alkyl groups; cyclic or non-cyclic alkyl groups; and mixtures thereof.

3. The process of claim 1, wherein the transition metal comprises at least one of palladium, platinum, nickel, ruthenium, cobalt, rhodium, rhenium, copper, and mixtures thereof.

4. The process of claim 1 wherein the transition metal is deposited onto the sulfonic acid functionalized SiO₂ carrier in an amount of from 0.01 to 20 percent by weight of the metallic compound based on the total weight of the heterogeneous catalyst.

5. The process of claim 1 where the molar ratio of transition metal to sulfur is in the range of 0.1 to 1.

6. The process of claim 1, wherein the ether is an unsymmetric ether, a symmetric ether or a cyclic ether.

7. The process of claim 1, wherein the process is a vapor-phase reduction process carried out under vapor-phase process conditions.

8. The process of claim 1, wherein the process is a liquid-phase reduction process carried out under liquid-phase process conditions.

9. A solvent comprising an ether produced from the process of claim 1.
